(19) United States
(12) Patent Application Publication
(10) Pub. No.: US 2025/0017633 A1
(43) Pub. Date: Jan. 16, 2025

(54) ACTUATED SIMULTANEOUS SEGMENTAL REDUCTION

(71) Applicant: GLOBUS MEDICAL, INC., AUDUBON, PA (US)

(72) Inventors: David Leff, Philadelphia, PA (US); Matthew Darnall, Chester Springs, PA (US)

(21) Appl. No.: 18/352,561

(22) Filed: Jul. 14, 2023

(51) Int. Cl. A61B 17/70

(52) U.S. Cl. CPC A61B 17/7086

(57) ABSTRACT

Reducer instruments, systems, and methods thereof for reducing a spinal rod. A series of reducers attachable to respective bone fasteners may be actuated by pneumatic or electronic control. Each reducer may have a reducer body having a pair of arms separated by a longitudinal slot configured to receive the spinal rod. A pusher may be slidably engaged with the arms with a distally extending tip configured to translate the spinal rod along the slot. The reducer may include a housing having a cylindrical body with a moveable component configured to drive the pusher to apply a compressive load to the spinal rod. For example, the moveable component may be piston or threaded rod configured to translate the pusher, and thereby drive the spinal rod into alignment.

### FIELD OF THE INVENTION

The present disclosure relates to orthopedic instruments, systems, and methods, and more particularly, to actuated reducer instruments configured to reduce a spinal rod into bone fasteners.

### BACKGROUND OF THE INVENTION

Many types of spinal irregularities can cause pain, limit range of motion, or injure the nervous system within the spinal column. These irregularities can result from, without limitation, trauma, tumor, disc degeneration, and disease. One general example of spinal irregularity is an abnormal curvature of the spine, for example, as exhibited with scoliosis, kyphosis, and/or lordosis. Treatment of irregular spinal curvatures can include, for example, reducing the severity and preventing further progression of the irregularity through physical therapy, bracing, and/or surgery. Surgical procedures can include realigning or correcting the curvature of the spine and optionally placing one or more rods alongside thereof to maintain the alignment.

The most common practice to correct or reduce a sagittal deformity of the spine is to contour a rod to a shape approximating the desired curvature of the spine. A segmental reducer is attached to a pedicle screw to apply a compressive load between the rod and the pedicle screw until the pedicle screw mates with the rod and the vertebra it is attached to has moved to the desired position. Reducers may be attached to each pedicle screw at each vertebra or segment. Surgeons may perform reduction segmentally, for example, reducing each vertebra individually to distribute the load across the spine, and sequentially by progressively tightening each reducer to achieve correction without overloading or pulling out the pedicle screw.

Such reducers and methods of reduction may have limitations, however, such as the tedious process of manually tightening each reducer, the load being unevenly distributed across the pedicle screws, the risk of accidentally overtightening the screw(s), and the possibility of screw pullout. As such, there exists a need for reduction devices and systems capable of addressing these limitations.

### SUMMARY OF THE INVENTION

To meet this and other needs, and in view of its purposes, the present application provides instruments, systems, and methods for reducing a deformity of the spine. In particular, a system including actuated reducers are attached to pedicle screws to apply a compressive load between the spinal rod and the pedicle screws. Instead of manual operation (e.g., manually tightening each reducer), the actuated reducers may be driven via electrical or pneumatic control. The reducers may be simultaneously driven instead of sequentially driven so that load can be evenly distributed across the pedicle screws. The actuated reducers may help to avoid the tedious process of manually tightening each reducer, lower the risk of accidentally over-tightening one or more screws, and/or minimize the likelihood of screw pullout.

According to one embodiment, a system for reducing a spinal rod includes a plurality of reducers configured to be actuated by pneumatic or electronic control. Each reducer may have a reducer body and a housing coupled to the reducer body. The reducer body may have a pair of arms separated by a longitudinal slot configured to receive the spinal rod. A pusher may be slidably engaged with the arms and may have a distally extending tip configured to translate the spinal rod along the slot. The housing may have a cylindrical body with a moveable component configured to drive the pusher to apply a compressive load to the spinal rod.

The system may include one or more of the following features. The system may further include a pneumatic or electronic control system including a panel having a plurality of knobs configured to control each reducer. The panel may include an inlet port and a plurality of outlet ports. The inlet port may be connected by pneumatic tubing or wiring to a supply of compressed air or electricity, and the outlet ports may be connected to the respective reducers. The control system may be a pneumatic system with a main regulator placed in series with regional regulators and segmental regulators connected in parallel by manifolds. The cylindrical body may be a pneumatic cylinder configured to receive compressed air. The moveable component may include a piston fitting snuggly inside the pneumatic cylinder, and the piston may be configured to translate the pusher distally to drive the spinal rod into alignment. The piston may include a piston head, a piston rod extending distally from the piston head, and a stem protruding proximally from the piston head. In another embodiment, instead of a piston, the reducer may also include a pneumatic motor coupled to a gearbox. The moveable component may include a threaded rod extending through the reducer body and a nut engaged with the threaded rod. The gearbox may be configured to drive the nut to, in turn, drive the threaded rod. Alternatively, the cylindrical body may include an electric motor. The moveable component may include a threaded rod extending through the reducer body and a nut engaged with the threaded rod. The electric motor may be configured to drive the nut to, in turn, drive the threaded rod.

According to another embodiment, an actuated reducer for reducing a spinal rod includes a pneumatic cylinder attached to a reducer body along a central tool axis. The reducer body has a pair of arms separated by a longitudinal slot configured to receive the spinal rod, and a pusher slidably engaged with the arms and having a distally extending tip configured to translate the spinal rod along the slot. The pneumatic cylinder defines a chamber and receives a piston therethrough. When compressed air is supplied to the chamber, the piston translates the pusher along the central tool axis to drive the spinal rod.

The actuated reducer may include one or more of the following features. The piston may be cannulated to define a central bore extending along the central tool axis configured to receive a locking cap driver. The piston may include a piston head, a piston rod extending distally from the piston head, and a stem protruding proximally from the piston head. A first ring seal may be positioned at a proximal end of the reducer and a second ring seal may be positioned within the piston head to seal the chamber and prevent air leakage. The pneumatic cylinder may include an input nozzle defining a through bore in fluid communication with the chamber of the pneumatic cylinder. The pusher may slide around the outside of the arms, and the pusher may be V-shaped with the distally extending tip having a concave recess configured to abut the spinal rod.

According to another embodiment, an actuated reducer for reducing a spinal rod includes a pneumatic motor attached to a reducer body. The reducer body is aligned along a reducer tool axis and the pneumatic motor is aligned along a motor axis offset relative to the reducer tool axis. The reducer body has a pair of arms separated by a longitudinal slot configured to receive the spinal rod, a pusher slidably engaged with the arms and having a distally extending tip configured to translate the spinal rod along the slot, a threaded rod extending through the reducer body, and a nut positioned at a proximal face of the reducer body. The pneumatic motor includes a motor and gearbox having a gear engaged with the nut. When compressed air is supplied to the motor, the gear rotates the nut, which drives the threaded rod, thereby translating the pusher to drive the spinal rod. In an alternative embodiment, the pneumatic motor may be replaced with an electric motor to rotate the nut and drive the threaded rod.

According to yet another embodiment, a method of correcting a spinal deformity may include one or more of the following steps in any suitable order: (1) anchoring a bone fastener in a vertebra, the bone fastener having a tulip head attached to a screw; (2)) positioning a spinal rod through the slot in the reducer body; (3) attaching a reducer to the tulip head of the bone fastener, the reducer including a reducer body having a pair of arms separated by a longitudinal slot, a pusher slidably engaged with the arms and having a distally extending tip; and (4) actuating the reducer via pneumatic or electronic control to move the pusher distally to translate the spinal rod along the slot and into the tulip head of the bone fastener. A plurality of bone fasteners may be attached to vertebrae and a plurality of reducers may be attached to the respective bone fasteners. The plurality of reducers may be actuated simultaneously to evenly distribute load across the bone fasteners. The plurality of reducers may be controlled by a robotic navigation system or may be controlled manually by a series of knobs.

Also provided are kits including reducers of varying types, a control panel including inlet and outlet tubing or wiring, spinal rods, fasteners or anchors, k-wires, insertion tools, and other components for performing the procedure.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 shows a pneumatic reducer attached to a vertebra for reducing a spinal rod into a tulip head of a pedicle screw according to one embodiment;

FIG. 2 shows a series of the pneumatic reducers as shown in FIG. 1 attached to vertebrae of the spine for reducing the spinal rod according to one embodiment;

FIG. 3 shows an operating set-up of a pneumatic or electrical reduction system with robotic or manual control according to one embodiment;

FIGS. 4A-4C show a pneumatic control apparatus system, the associated plumbing, and one pressure regulator, respectively, according to one embodiment;

FIGS. 5A-5C show front views of the reducer system at the start of reduction, partially reduced, and with biased reduction pressure, respectively, according to one embodiment;

FIGS. 6A-6B show front and cross-sectional views, respectively, of the pneumatic cylinder reducer with an internal piston according to one embodiment;

FIGS. 7A-7B show front and cross-sectional views, respectively, of an actuated reducer having a pneumatic motor for driving a threaded rod and nut according to one embodiment;

FIGS. 7C-7D show top and wireframe perspective views, respectively, of the pneumatic motor reducer and header for the actuated reducer of FIGS. 7A-7B according to one embodiment;

FIG. 8 shows a front view of an electric motor reducer according to one embodiment;

FIG. 9 shows a navigated pneumatic cylinder reducer having tracking markers according to one embodiment; and

FIG. 10 shows an encoded pneumatic cylinder reducer section according to one embodiment.

### DETAILED DESCRIPTION OF THE INVENTION

Embodiments of the disclosure are generally directed to instruments, systems, and methods for reducing spinal rods to provide correction, stability, and support during spinal fusion surgery. Specifically, actuated reducers are attached to pedicle screws to apply a compressive load between the spinal rod and the pedicle screws. Instead of manually tightening each reducer, the actuated reducers are driven via electrical or pneumatic control. Multiple actuated reducers allow for simultaneous reduction of the spine which may help to provide even distribution or bias of reduction forces. Methods for controlling the reduction forces are also provided to persuade the spinal rod into the desired position. Although described with reference to reducing a spinal rod during spinal surgery, it will be appreciated that the instruments and systems described herein may also be used in other orthopedic or trauma applications.

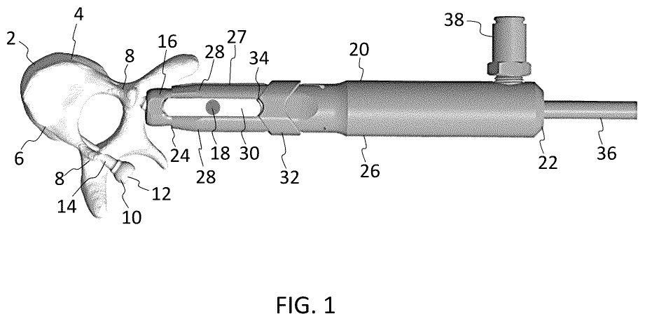

Referring now to FIGS. 1-3, a system of actuated reducers 20 for reducing a spinal rod 18 is shown according to one embodiment. In FIG. 1, an axial or overhead view of the spine 2 showing vertebra 4 including vertebral body 6 is provided. During spinal surgery, one or more bone fasteners 10 may be anchored into the vertebrae 4. The bone fasteners 10 may be installed during a minimally invasive surgical (MIS) procedure, for example, such that the bone fastener 10 is positioned through a guide tube or cannula to access and guide the bone fastener into the vertebra 4. In this embodiment, the bone fasteners 10 include pedicle screws anchored into the pedicles 8 of the vertebra 4, which are small bony structures connecting the vertebral body 6 to the facet joints in the spine 2. The pedicle screws 10 create solid anchor points for the spinal rod 18. The final construct helps to provide correction, facilitate the fusion process, and offer stability to the spine 2.

Each pedicle screw 10 includes an enlarged head 12 and a threaded shank or shaft 14 extending therefrom. The screw head 10 may be partially or fully rounded, spherical, or otherwise configured to interface with a tulip element or tulip head 16, which may be modular. The tulip head 16 may include a pair of arms defining a U-shaped channel sized and dimensioned to receive the spinal rod 18 therein. The tulip head 16 may be configured to pivot or rotate relative to the screw 10 enabling the spinal rod 18 to be inserted at various angles and optimize alignment. Once the spinal rod 18 is fully seated in the tulip head 16, the tulip head 16 is configured to secure the spinal rod 18 therein, for example, with a locking cap 17 or other securing mechanism. Examples of bone fasteners, other implants, and rod constructs are described in more detail, for example, in U.S. Pat. No. 10,388,917, which is incorporated by reference herein in its entirety for all purposes. Although pedicle screws are exemplified herein, it will be appreciated that the bone fasteners may include other bone screws, anchors, clamps, or the like configured to anchor to bone.

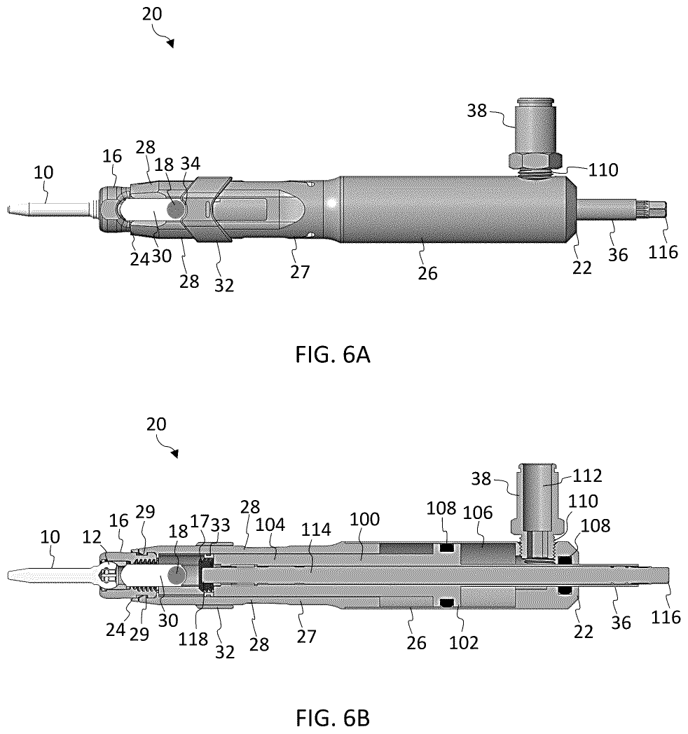

The actuated reducer 20 is configured to temporarily attach to the tulip head 16 to reduce the spinal rod 18 therein. The actuated reducer 20 may extend between a proximal end 22 and a distal end 24 along a central tool axis. The reducer 20 includes a housing or outer casing 26 having a generally tubular or cylindrical body. In one embodiment, the proximal portion of the outer casing 26 is a pneumatic cylinder. The outer casing 26 may be attached to or integral with a reducer body 27. The reducer body 27 includes a pair of elongated arms 28 separated by a longitudinal slot 30 configured to receive the tulip head 16 between the distal ends of the two arms 28. The spinal rod 18 is also translatable along the slot 30 along the central tool axis. The arms 28 may be arranged in parallel to one another. As best seen in FIG. 6B, the arms 28 may include inwardly projecting protrusions 29 receivable in corresponding recesses in the outside of tulip head 16. It will be appreciated that any compatible mating surfaces or interfaces may be used to engage and temporarily secure the reducer 20 to the tulip head 16.

A reduction member or pusher 32 may be slidably engaged with the reducer 20. The pusher 32 may be a tubular member having a cannula extending therethrough. The pusher 32 may have an outer body positioned around the outside of the reducer 20. In some embodiments, the pusher 32 may be generally chevron-shaped or V-shaped when viewed from the side. The pusher 32 includes a distally extending tip 34, which may include a partially circular or concave cut-out configured to engage or abut the spinal rod 18. In one embodiment shown in FIG. 6B, the pusher 32 may include an internal ledge 33, ring, or other member configured to mate or interface with a moveable drive component. The pusher 32 may be configured to slide longitudinally (e.g., distally and/or proximally), when forced by the moveable drive component. The pusher 32 translates along the reducer body 27 to translate the spinal rod 18 into the desired position. In one embodiment of a pneumatic system shown with reference to FIGS. 6A-6B, the moveable drive component is an internal piston 100. The pusher 32 may be translated by the piston 100, which is configured to press against ledge 33 to drive pusher 32 distally. The piston 100 may also include a hollow stem 36, which protrudes proximally from the pneumatic cylinder 26. The stem 36 may translate or slide longitudinally as the piston 100 moves through the reducer 20.

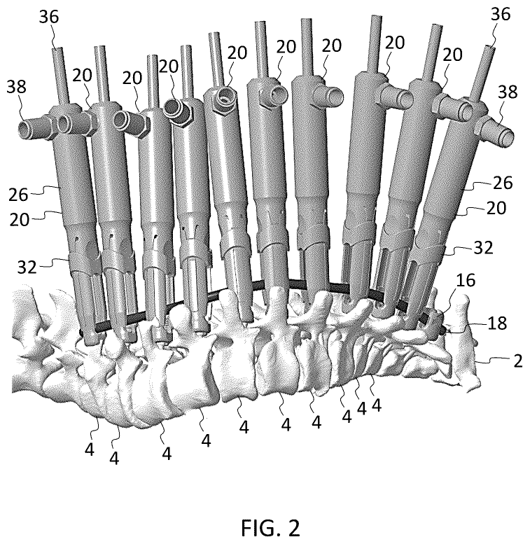

As best seen in the embodiment shown in FIG. 2, a series of actuated reducers 20 may be attached to multiple pedicle screws 10 secured to vertebrae 4 of the spine 2. Any suitable number of reducers 20 may be selected by the surgeon to perform the desired correction. After the pedicle screws 10 are anchored to the vertebrae 4, the actuated reducers 20 may be temporarily affixed to the tulip heads 16 of the screws 10. The spinal rod 18 may be positioned before or after attachment of the reducers 20. The spinal rod 18 extends through the respective slots 30 of the reducers 20 and is configured to be contoured and/or moved into position by the reducers 20. To reduce the spinal rod 18 and urge the spinal rod 18 into secure engagement with the tulip head 16, the pushers 32 are translated distally. As each pusher 32 translates distally, it may apply a radial force on arms 28, compressing the slot 30 and causing the arms 28 to clamp the tulip head 16 therebetween. The pusher 32 may be translated distally until the pusher 32 abuts the spinal rod 18 and urges or pushes the spinal rod 18 distally and into engagement with the tulip head 16. In one embodiment, all actuated reducers 20 may be simultaneously driven so that load can be evenly distributed across all of the pedicle screws 10.

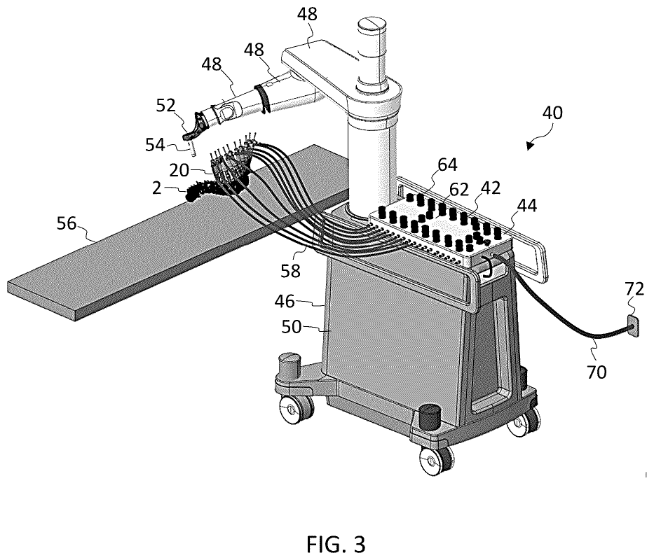

Each actuated reducer 20 may be controlled via an electrical and/or pneumatic control system 40. The control system 40 may include an electrical or pneumatic distribution and control apparatus 42 and a user interface system 44 either with manual dials or knobs 64 or controlled through a robotic and/or navigated system 46. FIG. 3 illustrates one embodiment of the actuated reducers 20 configured to be controlled by the robot system 46 and/or by manual dials or knobs 64.

The surgical robot and/or navigation system may include, for example, surgical robot 46 having one or more robot arms 48; a base 50 on wheels containing one or more computers having a processor, programming, and/or memory; a display, monitor, and/or optional wireless tablet (not shown) electronically coupled to the computer; an end-effector 52 including a guide tube 54 configured to receive and orient a surgical instrument, the end-effector 52 being electronically coupled to the computer and movable via arms 48 controlled by at least one motor based on commands processed by the computer; and/or one or more tracking markers. The surgical robot and/or navigation system may also utilize a camera (not shown), for example, positioned on a camera stand to move, orient, and support the camera in a desired position. The camera may include any suitable camera, such as one or more infrared cameras (e.g., bifocal or stereophotogrammetric cameras), able to identify and track, for example, active and passive tracking markers in a given measurement volume. The system may further include 2D & 3D imaging software that allows for preoperative and intraoperative planning, navigation, and guidance. Further examples of surgical robotic and/or navigation systems can be found, for example, in U.S. Pat. Nos. 10,675,094 and 9,782,229, which are incorporated by reference herein in their entireties for all purposes.

FIG. 3 illustrates a potential configuration for the placement of the surgical robot system in an operating room environment. For example, the robot 46 may be positioned near or next to a patient. The patient may be positioned on an operating table 56 and the spine 2 may be accessed in a suitable surgical manner. Once the pedicle screws 10 are anchored and the actuated reducers 20 are attached, the reducers 20 may be connected to the electrical and/or pneumatic control system 40. For example, a wire (if electrical) or tube (if pneumatic) 58 may be connected from each electrical or pneumatic connection or input 38 on the reducer 20 to a corresponding outlet port 68 on the distribution and control apparatus 42. Alternatively, the reducers 20 may be pre-connected to the control system 40 prior to attachment to the tulip heads 16. In the case of an electrical system, each wire 58 may be supplied with electrical power or electrical signals necessary to control each respective actuated reducer 20. In the case of pneumatic system, each tube 58 may be supplied with gases (e.g., compressed air) or fluids configured to control each respective actuated reducer 20.

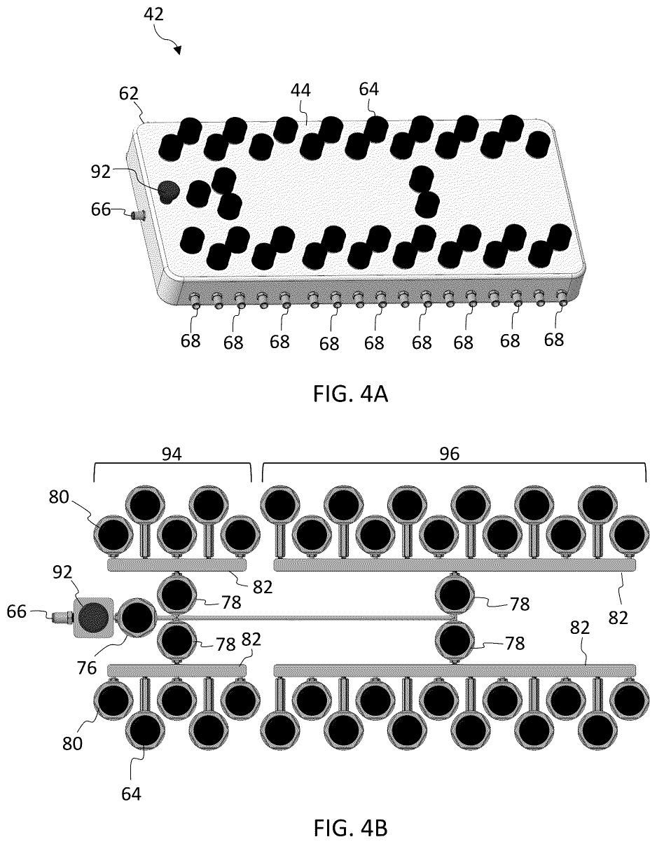

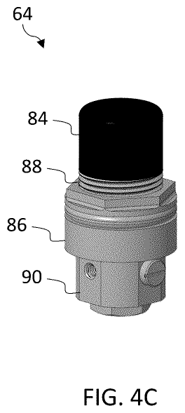

With further emphasis on FIGS. 4A-4C, one embodiment of the control system 40 configured for pneumatic distribution is shown. The pneumatic control apparatus 42 may include a panel 62 with user interface 44 having a plurality of knobs 64 configured to be manipulated manually by the user or controlled through robotic and/or navigated system 46. The panel 62 has an inlet port 66 and a plurality of outlet ports 68 arranged around the side of the panel 62. The inlet port 66 may be connected by pneumatic tubing 70 to a wall outlet 72, compressor, or other suitable source and supplied with a constant supply pressure of compressed air. The outlet ports 68 may be connected by pneumatic tubing 58 to the respective actuated reducers 20.

FIG. 4B depicts one example of pneumatic control apparatus plumbing. A main regulator 76 may be placed in series with regional regulators 78 and segmental regulators 80, which may be connected in parallel by manifolds 82. As shown, two sets of regional regulators 78, segmental regulators 80, and manifolds 82 may be mirrored to one another. An emergency stop vent 92 may be configured to open the system to release air pressure. The emergency stop 92 may be positioned before the main regulator 76. Knobs 64 or motors may be rotated to adjust the pressure regulator. As best seen in FIG. 4C, the knobs 64 may include control knobs or valve knobs that control the flow, pressure, or direction of the compressed air within the pneumatic system. Each knob 64 may include an adjustable grip 84 and a body 86 housing a spring cage 88, an internal adjusting screw assembly with an adjusting spring, and an inner valve assembly. A bottom plug 90 may complete the assembly. Although one set-up of the regulators 76, 78, 80 is shown, it will be appreciated that any suitable configuration may be used to regulate the respective reducers 20.

During operation, the user may be able to open or close the valves, increase or decrease the flow rate, or adjust the pressure level according to the desired settings. In this embodiment, every pedicle has segmental pressure regulators 80 connected in series to a regional lumbar regulator 78 or a regional thoracic regulator 78 for the left and right sides. This allows the user independent left-right control, regional control, and segmental control. Various configurations of the number and type of regulators, plumbing, and pressure regulators connected in series or parallel may provide more control options or simplify plumbing. It will be appreciated that the user interface 44 may be controlled manually by an operator or may be controlled by motors or other control systems. In an exemplary embodiment, the user operates the pneumatic user interface 44 via robot system 46.

During the reduction procedure, the compressive reduction load is applied and distributed by individually or collectively actuating the reducers 20. A single reducer 20 may be actuated to increase its load and further reduce the spine 2, or groups of reducers 20 may be simultaneously actuated to simultaneously increase load on screws 10 to distribute load. The amount that each reducer 20 is actuated may be biased such that load is increased more slowly on screws 10 with less pullout strength.

The series of reducers 20 may be actuated as desired to obtain the optimal performance. For example, the reducers 20 may be actuated simultaneously, sequentially, individually, or in any suitable order to achieve the desired correction. The pneumatic cylinders and motors may be controlled by regulating the pressure supplied to the input 38 of each reducer 20. Supplying the same pressure to each reducer 20 results in even distribution of reduction forces. Increasing this pressure results in increased reduction loads. As the pressure increases, additional air fills the cylinder or turns the pneumatic motor, actuating the reducer 20 and reducing the spine 2 until no further reduction can be achieved.

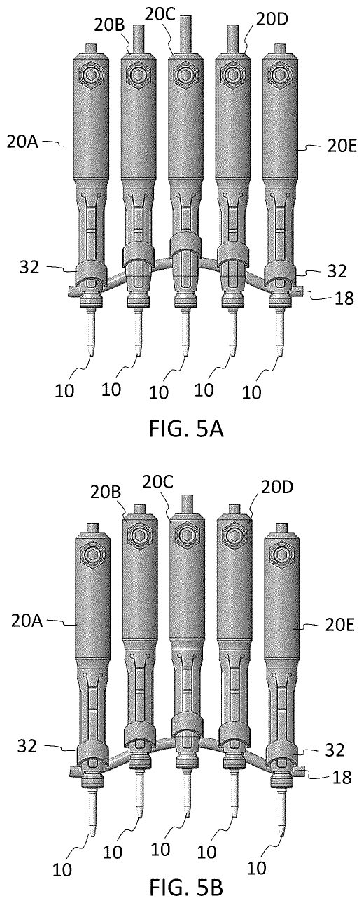

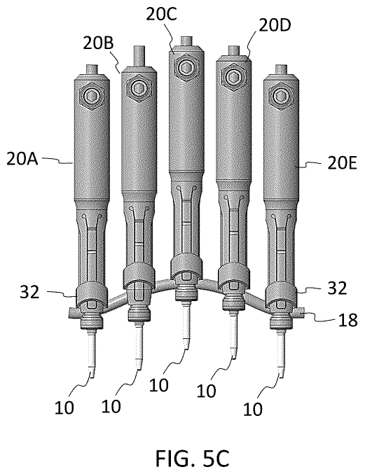

Turning now to FIGS. 5A-5C, examples of reduction with a series of five reducers 20A-20E is shown. FIG. 5A shows the beginning of reduction and FIG. 5B shows the reducers 20A-20E partially reduced. Outside reducers 20A, 20E start fully reduced and cannot be further displaced. The outer-middle reducers 20B, 20D have compressed until the spinal rod 18 was fully captured and cannot be reduced further. The middle reducer 20C requires additional reduction force, and increasing the pressure across the system will result in its reduction. FIG. 5C shows a biased reduction pressure. To bias reduction force away from pedicle screws 10 that may have decreased bone purchase, pressure may be unevenly distributed to the reducers 20. If the left-middle reducer 20B is supplied with less pressure, then it will apply less force than adjacent reducers 20A, 20C, resulting in it being reduced later in the reduction sequence at a decreased force.

The amount of pressure to each reducer 20 may be regulated by the distribution and control apparatus 42. In one embodiment, the plumbing configuration is configured to influence the pressure across the series of reducers 20A-20E. Adjustment of one or more pressure regulators results in a change in pressure in other downstream pressure regulators connected in series. For example, an increase in the main pressure regulator 76 results in the increase of the pressure in the regional and segmental pressure regulators 78, 80, resulting in a uniform increase in pressure across all reducers 20. However, adjustment of pressure regulators in parallel results in a difference in pressure between regulators. For example, decreasing the pressure regulator of the left-middle reducer 20B shown in FIG. 5C biases the applied pressure so that when the main pressure regulator 76 is opened, biased reduction force is applied. It will be appreciated that the pressure and forces applied to the reducers 20 may be influenced by any suitable type and configuration of control.

Turning now to FIGS. 6A-6B, actuated reducer 20 is shown in more detail and is configured for pneumatic operation according to one embodiment. The pneumatic cylinder 26 is rigidly attached to the reducer body 27 along the central tool axis. The actuated reducer 20 includes internal piston 100 configured to translate pusher 32 along the tool axis to drive spinal rod 18 into alignment. The piston 100 includes a piston head 102 and a piston rod 104. The stem 36 protrudes proximally from the piston head 102. The piston 100 converts energy from compressed air into linear motion. The piston head 102 fits snuggly inside the pneumatic cylinder 26. The pneumatic cylinder 26 defines an internal chamber or central bore 106 extending longitudinally along the central tool axis configured to receive the piston 100 therein. The piston 100 runs through the pneumatic cylinder 26 with piston seals 108 at either end. For example, ring seals 108 or other suitable seal may be provided at proximal end 22 and within piston head 102 to seal the chamber 106 and prevent air leakage. When compressed air is supplied to the chamber 106, the piston 100 translates the pusher 32 along the central tool axis, the pusher 32 abuts the spinal rod 18 and drives it distally.

The input nozzle 38 may be secured to the proximal end 22 of the pneumatic cylinder 26. The input nozzle 38 may include a threaded interface 110 to secure the input nozzle 38 to the pneumatic cylinder 26. The input nozzle 38 defines a through-bore or inner diameter 112 that intersects the inner bore 106 of the pneumatic cylinder 26. The axis of through-bore 112 of input nozzle 38 may be generally perpendicular to the axis of bore 106 through cylinder 26. The pneumatic hose or air tubing 58 is connected to the free end of the input nozzle 38. When compressed air is supplied through input nozzle 38 and into the proximal side of the cylinder 26, the air exerts pressure on the piston head 102, thereby moving piston 100 including piston head 102, piston rod 104, and proximal stem 36. The piston's movement is transferred to the attached pusher 32, causing the pusher 32 to move distally. The input pressure creates a pressure difference across the head 102 of the piston 100 to force pusher 32 against the spinal rod 18 and move the spinal rod 18 into the desired position.

The pneumatic cylinder force calculation may be represented by Equation (1) below. The amount of force applied (Fs) is equal to the pressure regulated by the system (Ps) multiplied by the pressurized surface area of the piston (As):

$${F}_{S} = {P}_{S} {A}_{S} \qquad Equation ( 1 )$$

This equation may be used to design a piston bore of sufficient size to apply a maximum amount of force required to reduce the spine, to govern the control system where the applied force is linearly proportional to the applied pressure, and can be adjusted between zero and the maximum available pressure.

The piston 100 may have a single-acting configuration, where air pressure is applied to only one side of the piston 100 (e.g., the proximal side), thereby allowing the piston 100 to move in one direction (e.g., distally). The piston 100 may be configured to return proximally through a spring or external force. In an alternative embodiment, the piston 100 may have a double-acting arrangement such that air pressure is applied alternately to both sides of the piston head 102, thereby allowing the piston 100 to move in both directions.

A locking cap driver 114 is positionable through the body of the reducer 20. The locking cap driver 114 runs centrally through a channel through the stem 36, piston head 102, and piston rod 104 of piston 100. The driver 114 has a proximal end 116 configured to interface with a handle (not shown) and a distal end 118 configured to engage and rotate the threaded locking cap 17. FIGS. 6A-6B show the actuated reducer 20 temporarily attached to the tulip head 16 of pedicle screw 10. After the spinal rod 18 has been fully reduced and inserted into the tulip head 16, the locking cap driver 114 allows the user to insert and tighten the locking cap 17 to secure the spinal rod 18 within the tulip head 16.

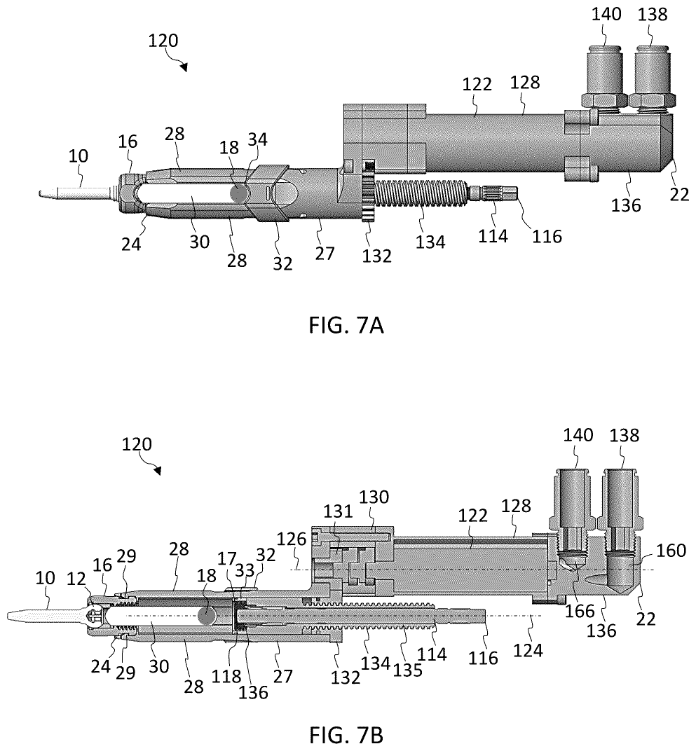

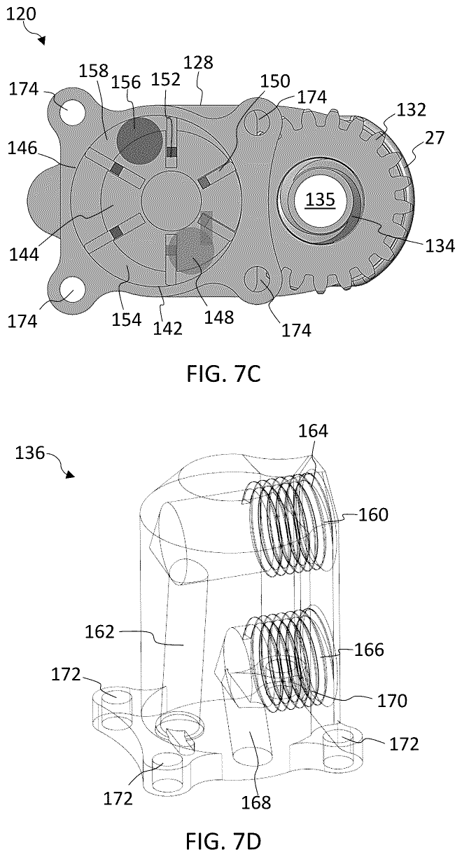

Turning now to FIGS. 7A-7D, an actuated reducer 120 configured for pneumatic operation is shown according to one embodiment. In this embodiment, actuated reducer 120 is similar to reducer 20, except the piston system is replaced with a pneumatic motor system. In particular, the actuated reducer 120 includes reducer body 27 attached to pneumatic motor 122, which controls movement of the pusher 32. The reducer body 27 is aligned along a first reducer tool axis 124, which aligns with the spinal rod 18 and tulip head 16. When forced by pusher 32, the spinal rod 18 translates along slot 30 and into tulip head 16. The pneumatic motor 122 is aligned along a second motor axis 126, which is offset relative to first axis 124. First and second axes 124, 126 may be generally parallel with one another.

The pneumatic motor 122 includes a casing or housing 128 having a generally tubular or cylindrical body. The pneumatic motor 122 may be coupled to a gearbox 130. The gearbox 130 may include a series of gears 131 that mesh together to transfer pneumatic power to mechanical power. The gearbox 130 has an output that drives a nut 132. The nut 132 may have gear-like teeth configured to mesh with one of the gears 131 in the gearbox 130. The nut 132 may be positioned at the proximal face of the reducer body 27. The nut 132 drives a threaded rod 134 extending through the reducer body 27. The threaded rod 134 may be aligned along reducer tool axis 124. A distal end 136 of threaded rod 134 compresses the pusher 32 against the spinal rod 18. The threaded rod 134 is cannulated with a central channel 135 configured to receive locking cap driver 114 therethrough. FIGS. 7A-7B show the actuated reducer 120 temporarily attached to the tulip head 16 of pedicle screw 10. After the spinal rod 18 has been fully reduced and inserted into the tulip head 16, the locking cap driver 114 may be rotated to tighten the locking cap 17 and secure the spinal rod 18 within the tulip head 16.

The proximal end 22 of the pneumatic motor 122 includes a header 136 configured to regulate the air through the motor 122. An input 138 and output 140 are attached to the header 136. Similar to pneumatic input nozzle 38, nozzles 138, 140 may include a hollow cylindrical body with a threaded interface configured to secure each nozzle 38 to the header 136. Pneumatic hose or air tubing 58 is connected to the free ends of each nozzle 138, 140. Pneumatic tubing 58 supplies input air pressure and an output to vent used air. In this embodiment, the output 140 may be connected to tubing in order to maintain a closed cycle within the sterile environment to minimize the potential for contamination and to reduce noise. It will be appreciated that the output 140 may be left open or a suppressor may be attached to reduce noise.

The working principle of one embodiment of a pneumatic vane motor 142 is described with reference to FIG. 7C. As shown in FIG. 7C, a top view of the pneumatic motor reducer 120 with the header 136 omitted for clarity is provided. A rotor 144 is housed offset to a larger bore 146 of the motor body 128. Input air pressure is applied across the input area 148 by the header 136. Vanes 150 translate outward to contact the larger bore 146 when the vane chambers 152 of the rotor 144 are pressurized, creating a sealed chamber 154 at elevated pressure. Air is evacuated at the output location 156 by the header 136, causing the sealed chambers 158 adjacent to the output 156 to have lower pressure than the input chambers. The pressure differential between chambers 154, 158 results in a torque applied to the rotor 144, driving the air motor. Although a vane motor 142 is exemplified, it will be appreciated that alternate types and configurations of air motors may be used in the pneumatic reducer 120.

With further emphasis on FIG. 7D, the header 136 includes one or more connecting bores or channels 160, 162, 166, 168 in fluid communication and connecting the nozzles 138, 140 to the rotor 144. For example, the header 136 may include an input bore 160 in fluid communication with a first connecting bore 162. The inlet of input bore 160 defines interior threads 164 configured to threadedly interface with corresponding threads on the inlet nozzle 138. The axis of the through-bore of the inlet nozzle 138 and the axis of the inlet bore 160 of the header 136 may be coaxial. The connecting bore 162 may be angled or sloped to connect with the input area 148 of the rotor 144. The header 136 may include an outlet bore 166 in fluid communication with a second connecting bore 168. The outlet of the outlet bore 166 defines interior threads 170 configured to threadedly engage corresponding threads on the outlet nozzle 140. The axis of the through-bore of the outlet nozzle 140 and the axis of the outlet bore 166 may be coaxial. The connecting bore 168 may be angled or sloped to connect with the output area 156 of the rotor 144. Openings 172 may be provided at four corners of the header 136, which are configured to align with corresponding openings 174 through a proximal face of the motor housing 128. The openings 172, 174 may each be secured together with threaded or non-threaded fasteners, such as screw, pins, or other securing members, thereby affixing the header 136 to the pneumatic motor 122.

The torque applied by the motor 142 is linearly proportional to the pressure regulated by the system, similar to the piston described above. This torque results in an applied reduction force after the gear and thread reductions. When compressed air is supplied through input nozzle 138, the air exerts pressure on the pneumatic motor 122, moving rotor 144 and gearbox 130, turning nut 132, moving threaded rod 134, and ultimately translating pusher 32 distally and forcing the spinal rod 18 into the tulip head 16.

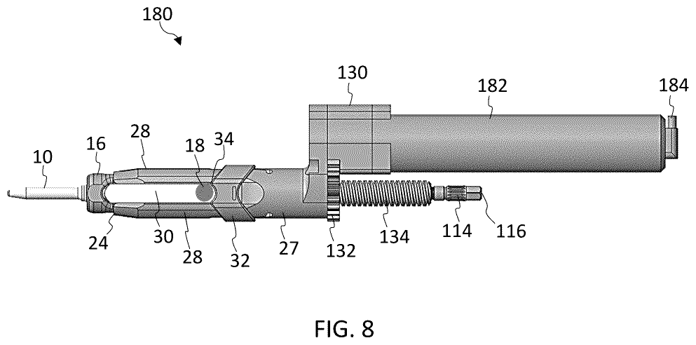

Turning now to FIG. 8, actuated reducer 180 configured for electric operation is shown according to one embodiment. In this embodiment, actuated reducer 180 is similar to reducer 120, except an electric motor 182 replaces the pneumatic motor. The reducer body 27 is controlled by the electric motor 182. Power and control cables 184 are attached to drive the motor 182. The motor 182 may include an induction motor, synchronous motor, brushed motor, brushless motor, stepper motor, or any other suitable motor. The electric motor 182 converts electrical energy into mechanical energy to gear box 130. Similar to reducer 120, gear box 130 rotates nut 132, driving threaded rod 134, thereby moving pusher 32 distally to translate spinal rod 18 along slot 30 and into tulip head 16.

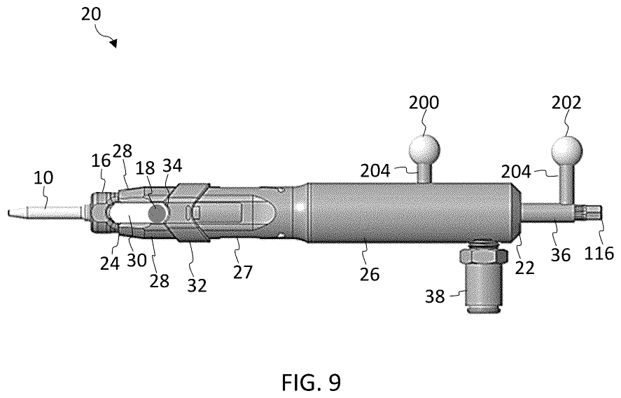

Turning now to FIG. 9, the actuated reducer 20, 120, 180 may be navigated by a navigation system. As described with reference to FIG. 3, the robotic system 46 or other suitable robot or navigation system may be used to monitor and control the actuated reducers 20, 120, 180. In this embodiment, the reducers 20, 120, 180 may be tracked by one or more fiducial markers or tracking markers 200, 202. The tracking markers 200, 202 may be viewable by the robot camera. The tracking markers 200, 202 may include reflective spheres configured to be tracked by the robot or navigation system. Although reflective spheres are exemplified in this embodiment, it will be appreciated that the fiducial markers may be replaced by active or passive markers or any suitable shape and type.

FIG. 9 depicts one embodiment of actuated reducer 20 including pneumatic cylinder 26 housing piston 100. A first tracking marker 200 may be attached to the pneumatic cylinder 26. The tracking marker 200 may be positioned on the outside of pneumatic cylinder 26, for example, via a first post 204. The tracking marker 200 may be positioned to extend outward in a direction opposite to the inlet nozzle 38. A second tracking marker 202 may be positioned on the stem 36 of the piston 100. Similarly, a second post 204 may be used to support the second tracking marker 202. The second tracking marker 202 is configured to move or translate in tandem with movement of stem 36 of piston 100. The first and second markers 200, 202 may be aligned along the same plane or may be otherwise configured. The navigation system may be configured to track movement of the first tracking marker 200 to monitor position and movement of the entire reducer 200. In addition, the navigation system may be configurated to track movement of the second tracking marker 202 relative to first tracking marker 200 to measure the distance piston 100 has translated or moved through the reducer 20. In this manner, the navigation system may be able to determine the position and movement of pusher 32 and corresponding placement of spinal rod 18.

Although two fiducial markers are exemplified, it will be appreciated that any suitable number, type, configuration, and placement of markers may be used to track the instrument and movement of spinal rod. Although described with reference to actuated reducer 20, it will be understood that the markers may also be applied to other actuated reducers or other types of instruments. For example, one or more markers may be affixed to the threaded rod 134 of the pneumatic reducer 120 or motor reducer 180 to enable a navigated system to measure the distance the reducer has actuated.

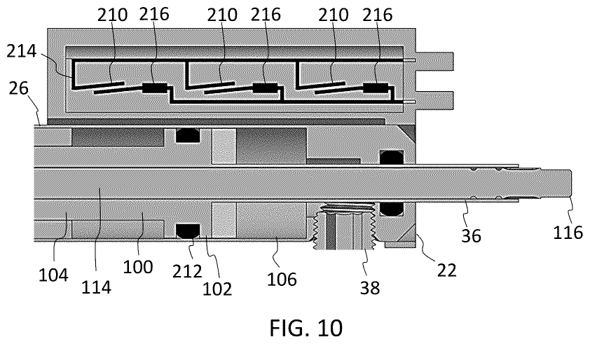

Turning now to FIG. 10, another system for monitoring an actuated reducer is shown. In this embodiment, reed switches 210 are placed along the length of the cylinder housing 26, which may be used to encode the position of the piston 100. When a magnet 212 attached to the head 102 of the piston 100 is translated adjacent to a pair of ferromagnetic wires 214 the gap is closed, resulting in the completion of the circuit. Resistors 216 are connected in series with the reed switch 210, and each unit of reed switch 210 and resistor 216 are connected in parallel such that the voltage drop across the circuit is encoded to the reed switch 210 being activated, and its position along the piston bore. This enables combed force-displacement control of reduction. The system may observe an increase in displacement of a single reducer, indicating that the screw 10 may be pulling out and bias the pressure regulator at that level to bias pressure away from that reducer. The system may also implement displacement control of reduction through dynamic control of applied forces. This may aid in mitigating displacement-related reduction complications, such as neurological deficits resulting from damage to the spinal cord or peripheral nerves.

The actuated reducers described herein may be actuated simultaneously, sequentially, individually, or in any suitable order to achieve the desired correction. In one embodiment, actuated reducers enable simultaneous reduction of the spine which allows for even distribution or bias of reduction forces and methods for controlling reduction forces through manual or electronic control. This enables a surgeon to more evenly distribute corrective forces across the spine and prevent screw pullout by biasing reduction forces away from screws with weaker purchase.

Instead of manually tightening each reducer, the actuated reducers may be driven via electrical and/or pneumatic control. Pneumatic systems maintain the sterile environment without complex and expensive sterile electronics which may deteriorate after multiple sterilizations. The compressibility of air and pneumatic distribution system act as a built-in safety system to prevent sudden overload and reduce control complexity compared to an electronic system. Electronic control of the pneumatic system or of electric motors enables control and distribution of forces by an electronic system to automate the correction process and enable the surgeon to focus on other tasks. The actuated reducers, pneumatic or electrical, may also be controlled with a robotic navigation system.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A system for reducing a spinal rod comprising:
a plurality of reducers configured to be actuated by hydraulic or electronic control,
each reducer having a reducer body and a housing coupled to the reducer body,
the reducer body having a pair of arms separated by a longitudinal slot configured to receive the spinal rod, a pusher slidably engaged with the arms and having a distally extending tip configured to translate the spinal rod along the slot, and
the housing having a cylindrical body with a moveable component configured to drive the pusher to apply a compressive load to the spinal rod.

2. The system of claim 1 further comprising a pneumatic or electronic control system including a panel having a plurality of actuators configured to control each reducer.

3. The system of claim 2, wherein the panel includes an inlet port and a plurality of outlet ports, the inlet port being connected by pneumatic tubing or wiring to a supply of compressed air or electricity, and the outlet ports being connected to the respective reducers.

4. The system of claim 2, wherein the control system is a pneumatic system with a main regulator placed in series with regional regulators and segmental regulators connected in parallel by manifolds.

5. The system of claim 1, wherein the cylindrical body is a pneumatic cylinder configured to receive compressed air.

6. The system of claim 5, wherein the moveable component comprises a piston fitting snuggly inside the pneumatic cylinder, and the piston is configured to translate the pusher distally to drive the spinal rod into alignment.

7. The system of claim 6, wherein the piston includes a piston head, a piston rod extending distally from the piston head, and a stem protruding proximally from the piston head.

8. The system of claim 1, wherein the reducer further comprises a pneumatic motor coupled to a gearbox.

9. The system of claim 8, wherein the moveable component comprises a threaded rod extending through the reducer body and a nut engaged with the threaded rod, wherein the gearbox is configured to drive the nut to, in turn, drive the threaded rod.

10. The system of claim 1, wherein the cylindrical body includes an electric motor.

11. The system of claim 10, wherein the moveable component comprises a threaded rod extending through the reducer body and a nut engaged with the threaded rod, wherein the electric motor is configured to drive the nut to, in turn, drive the threaded rod.

12. An actuated reducer for reducing a spinal rod comprising:
a pneumatic cylinder attached to a reducer body along a central tool axis,
the reducer body having a pair of arms separated by a longitudinal slot configured to receive the spinal rod, a pusher slidably engaged with the arms and having a distally extending tip configured to translate the spinal rod along the slot, and
the pneumatic cylinder defines a chamber and receives a piston therethrough, wherein when compressed air is supplied to the chamber, the piston translates the pusher along the central tool axis to drive the spinal rod.

13. The reducer of claim 12, wherein the piston is cannulated and defines a central bore extending along the central tool axis configured to receive a locking cap driver.

14. The reducer of claim 12, wherein the piston includes a piston head, a piston rod extending distally from the piston head, and a stem protruding proximally from the piston head.

15. The reducer of claim 14, wherein a first ring seal is positioned at a proximal end of the reducer and a second ring seal is positioned within the piston head to seal the chamber and prevent air leakage.

16. The reducer of claim 12, wherein the pneumatic cylinder includes an input nozzle defining a through bore in fluid communication with the chamber of the pneumatic cylinder.

17. The reducer of claim 12, wherein the pusher slides around an outside of the arms, and the pusher is V-shaped with the distally extending tip having a concave recess configured to abut the spinal rod.

18. A method of correcting a spinal deformity, the method comprising:
anchoring a bone fastener in a vertebra, the bone fastener having a tulip head attached to a screw;
attaching a reducer to the tulip head of the bone fastener, the reducer including a reducer body having a pair of arms separated by a longitudinal slot, a pusher slidably engaged with the arms and having a distally extending tip;
positioning a spinal rod through the slot in the reducer body; and
actuating the reducer via pneumatic or electronic control to move the pusher distally to translate the spinal rod along the slot and into the tulip head of the bone fastener.

19. The method of claim 18, wherein a plurality of bone fasteners are attached to vertebrae and a plurality of reducers are attached to the respective bone fasteners, and wherein the plurality of reducers are actuated simultaneously to evenly distribute load across the bone fasteners.

20. The method of claim 19, wherein the plurality of reducers are controlled by a robotic navigation system.
